(19) United States
(12) Patent Application Publication
(10) Pub. No.: US 2010/0081162 A1
(43) Pub. Date: Apr. 1, 2010

(54) METHOD OF EVALUATING ANTIWRINKLE SUBSTANCE AND METHOD OF ASSESSING THE SKIN

(75) Inventors: Naoko Kida, Kanagawa (JP); Akihiro Tada, Kanagawa (JP)

Correspondence Address: KNOBBE MARTENS OLSON & BEAR LLP, 2040 MAIN STREET, FOURTEENTH FLOOR, IRVINE, CA, 92614, US

(73) Assignee: POLA CHEMICAL INDUSTRIES INC., SHIZUOKA (JP)

(21) Appl. No.: 12/529,505

(22) PCT Filed: Feb. 18, 2008
(86) PCT No.: PCT/JP2008/052626

§ 371 Date: Sep. 1, 2009

(30) Foreign Application Priority Data
Mar. 1, 2007 (JP) 2007-051025

(51) Int. Cl. C12Q 1/02; C07D 311/02

(52) U.S. Cl. 435/29; 549/398

(57) ABSTRACT

It is intended to provide a method whereby the skin conditions (for example, loss in the skin elasticity, wrinkle formation possibility, insufficient tightness in dermal collagen fiber bundles, or the like) can be exactly and conveniently assessed. It is also intended to provide a method whereby the wrinkle-improvement effect of a test substance can be exactly and conveniently evaluated. The skin conditions are assessed by using the expression amount of an adhesion factor in skin cells as an indication. The wrinkle reducing effect of a test substance is evaluated by using the expression amount of an adhesion factor in skin cells in the presence of the test substance as an indication.

### TECHNICAL FIELD

The present invention relates to a method of evaluating an antiwrinkle substance. The present invention also relates to a method of assessing the skin.

### BACKGROUND ART

The skin suffers a variety of damages due to photo stimulation such as ultraviolet radiation, physical stimulation caused by environmental change such as seasonal or climatic change, chemical stimulation caused by chemical compounds such as detergents, and the like, and accumulates the damage with aging.

The epidermis and dermis of the skin include epidermal cells, fibroblasts, and extracellular matrices such as elastin and collagen which are present outside the cells and support the skin structure. With aging, the growth of fibroblasts is reduced, and the production amount of collagen decreases. As a result, skin elasticity and moisturizing function are deteriorated to thereby decrease skin tone and elastic force. Further, it has been reported that collagen fibers are denatured and altered with aging to cause loss in skin elasticity and cause wrinkle formation. In addition, there is known that the skin condition such as wrinkle or skin roughness and the degree of orderliness of dermal collagen fiber bundles are associated with each other (for example, see Patent Document 1).

As a method of evaluating a skin elasticity, there are known, a method of assessing disarrangement of dermal collagen fiber bundles by using, as an index, a characteristic of stress change against compression by external force in a skin surface (Patent Document 2) and a method of evaluating skin flexibility and elasticity by using an oxidized protein in the honey layer as an index (Patent Document 3), for example.

By a cell adhesion factor (hereinafter, also referred to as “adhesion factor”) is meant a membrane protein responsible for cell adhesion (cell adhesion molecule: CAM). Cadherin involved in formation and retention of adherens junction and integrin involved in cell-substrate adhesion are typical adhesion factors. As the adhesion factor, the following is also known: claudin present in tight junction; immunoglobulin superfamily molecules involved in cell-cell recognition in the epithelium, blood vessel endothelium, immune systems, nerves, and the like; selectin involved in tissue distribution of white blood cells; and neuroligin involved in nerve synapse induction, for example.

It has been reported that the amount of those adhesion factors decreases with cell senescence, and reduction in the amount of the adhesion factors leads to reduction in strength of cell-cell or cell-substrate bonding (Non-Patent Document 1).

On the other hand, Fucus vesiculosus, Laminaria japonica, Sargassum fusiforme, and the like are known as an accelerator for expressing integrin, for example (Patent Document 4). There is also known that skin cosmetics including those components and actin-myosin activators in combination improve skin wrinkle and sagging skin (Patent Document 5).

However, there is not known the relationship between the expression amount of the adhesion factor such as integrin and the loss in skin elasticity, wrinkle formation possibility, or insufficient tightness in dermal collagen fiber bundles.

Patent Document 1: WO 98/040045

Patent Document 2: JP 2004-089617 A

Patent Document 3: JP 2006-349372 A

Patent Document 4: JP 1.1-246428 A

Patent Document 5: JP 2001-278769 A

Non-patent Document 1: J. Photobiol B 27(1) P39-46 (1995)

### DISCLOSURE OF THE INVENTION

It is an object of the present invention to provide a method of evaluating a test substance for its wrinkle-improvement effect exactly and conveniently. It is another object of the present invention to provide a method of assessing a skin condition such as loss in skin elasticity, wrinkle formation possibility, or insufficient tightness in dermal collagen fiber bundles exactly and conveniently.

The inventors of the present invention have found that the tightness in dermal collagen fiber bundles is affected by the expression amount of the adhesion factor in the skin cells. Further, the inventors have found that various substances may be evaluated for their wrinkle-improvement effects by using, as an index, the expression amount of the adhesion factor in the cells, and thus, have completed the present invention. In addition, the inventors have found that a skin condition may be assessed by using the expression amount of the adhesion factor in the cells as an index, and thus, have completed the present invention.

In other words, the present invention is as follows.

The present invention provides:

(1) a method of evaluating an antiwrinkle substance, including evaluating a test substance for its wrinkle-improvement effect by using, as an index, an expression amount of an adhesion factor in an animal cell in the presence of the test substance;

(2) a method of evaluating an antiwrinkle substance according to Item (1), including evaluating a test substance for its wrinkle-improvement effect by using, as an index, a degree of a difference in expression amounts of an adhesion factor in an animal cell between in a presence and absence of the test substance;

(3) a method of evaluating an antiwrinkle substance according to Item (1), including evaluating a test substance exhibiting a larger difference in expression amounts of the adhesion factor to have a larger wrinkle-improvement effect;

(4) a method of evaluating an antiwrinkle substance according to Item (1), including evaluating a test substance in which the expression amount of the adhesion factor in the presence of the test substance is 1.5 or more times higher than the expression amount of the adhesion factor in the absence of the test substance to have a larger wrinkle-improvement effect;

(5) a method of evaluating an antiwrinkle substance according to Item (1), in which the adhesion factor is integrin;

(6) a method of assessing a skin, comprising assessing a skin condition by using, as an index, an expression amount of an adhesion factor in a skin cell;

(7) a method of assessing a skin according to Item (6), comprising assessing a skin exhibiting the lower expression amount of the adhesion factor to be in a poorer condition;

(8) a method of assessing a skin condition according to Item (7), comprising assessing the skin exhibiting the lower expression amount of the adhesion factor to have a larger loss in elasticity;

(9) a method of assessing a skin condition according to Item (7), comprising assessing a skin exhibiting the lower expression amount of the adhesion factor to have a higher wrinkle formation possibility;

(10) a method of assessing a skin condition according to Item (7), comprising assessing a skin exhibiting the lower expression amount of the adhesion factor to have a more insufficient tightness in dermal collagen fiber bundles;

(11) a method of assessing a skin condition according to Item (6), in which the adhesion factor is integrin;

(12) an external preparation for skin, containing an antiwrinkle substance evaluated to have a larger wrinkle-improvement effect by the method of evaluating an antiwrinkle substance according to Item (4); and

(13) a method of producing an external preparation for skin, comprising: evaluating a wrinkle-improvement effect by the method of evaluating an antiwrinkle substance according to Item (4); and formulating an antiwrinkle substance evaluated to have a larger wrinkle-improvement effect into an external preparation for skin.

### BRIEF DESCRIPTION OF THE DRAWINGS

FIG. 1 is a graph illustrating an expression amount of an integrin gene in an integrin (ITGA2) knockdown strain.

FIG. 2 is a graph illustrating an expression amount of an integrin gene in an integrin (ITGA5) knockdown strain.

FIG. 3 is a graph illustrating an expression amount of an integrin gene in an integrin (ITGB1) knockdown strain.

FIG. 4 is a photograph showing a cell shape of MOCK after 1 hour of adhesion (drawing-substituting photograph).

FIG. 5 is a photograph showing a cell shape of MOCK after 3 hours of adhesion (drawing-substituting photograph).

FIG. 6 is a photograph showing a cell shape of the integrin (ITGA2) knockdown strain after 1 hour of adhesion (drawing-substituting photograph).

FIG. 7 is a photograph showing a cell shape of the integrin (ITGA2) knockdown strain after 3 hours of adhesion (drawing-substituting photograph).

FIG. 8 is a photograph showing a shape of the integrin (ITGA5) knockdown strain after 1 hour of adhesion (drawing-substituting photograph).

FIG. 9 is a photograph showing a shape of the integrin (ITGA5) knockdown strain after 3 hours of adhesion (drawing-substituting photograph).

FIG. 10 is a photograph showing a shape of the integrin (ITGB1) knockdown strain after 1 hour of adhesion (drawing-substituting photograph).

FIG. 11 is a photograph showing a shape of the integrin (ITGB1) knockdown strain after 3 hours of adhesion (drawing-substituting photograph).

FIG. 12 are photographs each for comparing tightness conditions in collagen fiber bundles with or without farrerol in the presence of heparin (drawing-substituting photograph).

FIG. 13 is photographs each showing the observations of a tightness condition in collagen fiber bundles in changing a farrerol concentration in the presence of heparin (drawing-substituting photograph).

FIG. 14 is a graph illustrating an effect of farrerol on an expression amount of integrin.

FIG. 15 is a graph illustrating an effect of farrerol on an expression amount of integrin.

FIG. 16 is a graph illustrating an effect of farrerol on an expression amount of integrin.

FIG. 17 is a graph illustrating an effect of farrerol on an expression amount of collagen.

FIG. 18 is a graph illustrating an effect of farrerol on an expression amount of collagen.

FIG. 19 are photographs each showing behavior of an integrin protein after 3 and 6 hours of adhesion (drawing-substituting photograph).

FIG. 20 are photographs each showing behavior of an integrin protein after 15 and 24 hours of adhesion (drawing-substituting photograph).

### BEST MODE FOR CARRYING OUT THE INVENTION

The method of evaluating an antiwrinkle substance of the present invention includes evaluating a test substance for its wrinkle-improvement effect by using, as an index, the expression amount of an adhesion factor in skin cells in the presence of the test substance.

The cells are not particularly limited as long as they are cells derived from animals. For example, skin cells such as honey cells, epithelial cells, or fibroblasts may be used. Of those, fibroblasts are preferred. The cells harvested from the skin of animals may be cultivated before use. However, there are established culture cell lines, which are commercially-available, and such commercially-available products may also be used. As such commercially-available products, “Normal human skin fibroblasts (primary culture)” manufactured by Sanko Junyaku Co., Ltd. and “Normal human fibroblasts” manufactured by KURABO INDUSTRIES LTD. may be preferably exemplified. In the case where culture cells are used, the expression amount of the adhesion factor in the cells is investigated in advance, and cells exhibiting the lower expression amount of the adhesion factor are selected from those cells, isolated, and cultivated before use. This is preferred in terms of selecting an excellent antiwrinkle substance. Preferable examples of the origin of the cells include a mouse, rat, rabbit, and human. The human is particularly preferred due to its small difference in animal species.

The adhesion factor may be one expressed in horny cells, epidermal cells, or fibroblasts. The adhesion factor may be any one of an adhesion factor responsible for cell-cell bonding or an adhesion factor responsible for cell-substrate (matrix) bonding. Further, the bonding constructed by the adhesion factor may be homo-type or hetero-type, and strength of the bonding is also not particularly limited.

Examples of the adhesion factor include an adhesion factor belonging to the cadherin superfamily such as classic cadherin, desmocollin, or desmoglein (collectively referred to as “cadherin” herein), an adhesion factor belonging to the integrin family (collectively referred to as “integrin” herein), and claudin. Integrin is preferably used in the method of evaluating an antiwrinkle substance of the present invention. Integrin is a collective term of transmembrane cell adhesion factors formed of heterodimer glycoproteins having α-subunit and β-subunit. Integlin recognizes and binds to a specific sequence such as an RGD (Arg-Gly-Asp) sequence that an extracellular matrix protein such as collagen, fibronectin, vitronectin, or laminin has, while binds to a cytoskeleton such as actin inside the cells. In the case where the expression amount of integrin is used as an index, at least one expression amount of α-subunit and β-subunit may be measured. For example, in assessing insufficient tightness in dermal collagen fiber bundles or wrinkle formation possibility, α2 protein, α5 protein, and β1 protein are preferably exemplified. Of those, α2 protein and β1 protein forming a collagen receptor VLA-2 are preferably exemplified.

The expression amount of the adhesion factor is measured after cultivating the cells under a certain condition. The condition for a medium and cultivation may be any condition generally used for the cells. For example, in the case where fibroblasts are cultivated, DMEM supplemented with 10% FBS (manufactured by GIBCO) is used as a medium, and the cells are seeded thereto in a volume of about 10³ cells/cm² to 10⁵ cells/cm², followed by cultivation with addition of a test substance. The relationship between the concentration and the wrinkle-improvement effect of the test substance may be evaluated by changing stepwise the addition concentration of the test substance. Cultivation may be preferably carried out until the number of cells can be increased appropriately and the expression amount of the cell adhesion factor becomes constant. As an exemplary guide, cultivation may be carried out for about 3 to 5 days. During this period, the medium is replaced with a fresh one, if required.

The expression amount of the adhesion factor may be measured by extracting RNA contained in the harvested cells and determining the transcription amount of an gene encoding for the adhesion factor by the RT-PCR method, for example. The expression amount of the adhesion factor may also be measured by determining the amount of the adhesion factor present inside and outside the cells with an antibody specifically binding to the adhesion factor. In the case where the antibody is used, a labeled antibody may be directly used for quantification, or a secondary antibody may be labeled and then the secondary antibody may be used for quantification (sandwich method).

The thus measured expression amount of the adhesion factor is used as an index to evaluate a test substance for its wrinkle-improvement effect. For example, during cell cultivation, time-dependent change in the expression amount of the adhesion factor may be measured to evaluate the test substance for its wrinkle-improvement effect.

The test substance may also be evaluated for its wrinkle-improvement effect by comparing difference in the expression amounts of the adhesion factor in the absence and presence of the test substance among a plurality of the test substances. Specifically, a test substance exhibiting the larger difference is evaluated to have the larger wrinkle-improvement effect.

Further, when the expression amount of the adhesion factor in the presence of a test substance is 1.5 or more times higher than the expression amount of the adhesion factor in the absence of the test substance, the test substance is evaluated to have a larger wrinkle-improvement effect. In particular, when at least one of the following criteria is satisfied: the expression amount of α2 protein in the presence of a test substance is higher than the expression amount of α2 protein in the absence of the test substance by a factor of 2.0 or more and preferably 2.5 or more; and the expression amount of β1 protein in the presence of a test substance is higher the expression amount of β1 protein in the absence of the test substance by a factor of 1.5 or more and preferably 1.8 or more, the test substance is evaluated to have a larger wrinkle-improvement effect. The test substance evaluated to have a larger wrinkle-improvement effect through the above-mentioned evaluation may be suitably incorporated into an external preparation for skin as an antiwrinkle substance. An example of such an antiwrinkle substance includes farrerol.

The method of assessing the skin of the present invention includes assessing the skin condition by using, as an index, the expression amount of the adhesion factor in the skin cells.

In the method of assessing the skin of the present invention, the skin cells refer to the cells harvested from the skin. As the cell species to be used for evaluation, fibroblasts are used most preferably, although honey cells and epithelial cells may also be used.

The honey cells obtained by detaching and harvesting multiple layers, for example, about six layers of the cells from the skin in the living body with a pressure-sensitive tape may be cultivated before use. The honey cells are easily harvested from a human living body.

The epithelial cells and fibroblasts may be obtained by: harvesting the skin tissue by a biopsy or the like; dispersing the cells from the skin tissue through collagenase treatment; collecting the dispersed cells by centrifugation, and cultivating the cells in an appropriate culture solution.

The adhesion factor is as mentioned above. Further, the expression amount of the adhesion factor may be measured by the above-mentioned method.

Subsequently, the skin condition is assessed by using the expression amount of the adhesion factor as an index. An example of the method of assessing the skin condition includes a method of comparing the expression amounts of the adhesion factor among a plurality of individuals. Specifically, the skin exhibiting the lower expression amount of the adhesion factor is assessed to be in the poorer condition. Further, the skin exhibiting the lower expression amount of the adhesion factor is assessed to have the larger loss in elasticity. In addition, the skin exhibiting the lower expression amount of the adhesion factor is assessed to have the higher wrinkle formation possibility. Yet in addition, the skin exhibiting the lower expression amount of the adhesion factor is assessed to have the more insufficient tightness in dermal collagen fiber bundles.

In this case, assessment becomes easier: by using the expression amount of the adhesion factor in average individuals of the same age or generation, or the expression amount of the adhesion factor in juvenile individuals as a positive control; by using the expression amount of the adhesion factor in senile individuals as a negative control; and by comparing the expression amounts of the adhesion factor at the ultraviolet radiation exposure site and non-exposure site in the same individual. Further, the accumulation and statistical processing of the assessment results allow classification such as ranking to thereby provide a higher commercial value to the method of assessing the skin of the present invention.

Further, time-dependent change in the expression amount of the adhesion factor in the same individual may be recorded to expect the transition of later skin condition. This is because change in the adhesion factor is followed by change in the skin condition.

### EXAMPLES

### Example 1

Hereinafter, the present invention is specifically described by way of examples, but the present invention is not limited thereto.

<Knockdown with siRNA>

In accordance with the procedure as described below, integrin genes of normal human fibroblasts (integrin α2 (ITGA2), integrin α5 (ITGA5), and integrin β1 (ITGB1)) are knocked down with siRNA, and the expression amounts of integrin in those knockdown strains were measured by the RT-PCR method.

<Cells and Reagents Used>

1) Cells and media

Normal human fibroblasts (Passage=3 was used in this examination) (KURABO)

10% FBS+DMEM (GIBCO)

2) siRNA

20 μM siRNA ITGA2 (DHARMACON)
D-004566-02, hereinafter, ITGA2(2)
D-004566-03, hereinafter, ITGA2(3)
D-004566-04, hereinafter, ITGA2(4)
D-004566-05, hereinafter, ITGA2(5)

20 μM siRNA ITGA5(DHARMACON)
D-008003-4, hereinafter, ITGA5(4)
D-008003-5, hereinafter, ITGA5(5)
D-008003-6, hereinafter, ITGA5(6)
D-008003-7, hereinafter, ITGA5(7)

20 μM siRNA ITGB1 (DHARMACON)
D-004506-01, hereinafter, ITGB1(1)
D-004506-02, hereinafter, ITGB1(2)
D-004506-03, hereinafter, ITGB1(3)
D-004506-04, hereinafter, ITGB1(4)
   3) Transfection reagent

Lipofectamine 2000 (Invitrogen, lipofection reagent)

OPTIMEM (Gibco)

4) RNA extraction reagent

RNeasy Mini Kit (50) (QIAGEN: Cat. No. 74104)

QIA shuredder™ (50) (QIAGEN: Cat. No. 79654)

RNase-Free DNase set (50) (QIAGEN: Cat. No. 79254)

5) Reverse transcription reaction reagent

SuperScript™ III platinum Two-Step qRT-PCR Kit with SYBR Green (Invitrogen: Cat. No. 11735-032)

6) Real-time PCR reagent

QuantiTect SYBR Green PCR Kit (QIAGEN: Cat. No. 204143)

### 7) PCR Primer

QuantiTect (registered trademark) Primer Assays
ACTB (QIAGEN: Cat. No. QT)
ITGA2 (QIAGEN: Cat. No. QT00086695)
ITGA5 (QIAGEN: Cat. No. QT00080871)
ITGB1 (QIAGEN: Cat. No. QT00068124)

<Protocol>

1-1) Transfection of siRNA
(1) 60 to 80% confluent NHDF cells were detached with 0.25% Trypsin/EDTA to prepare a cell suspension of 2.5×10⁴ cells/ml.
(2) The cell suspension in (1) was seeded into a 12-well plate in a volume of 2 ml/well. Incubation was carried out in 5% CO₂ at 37° C. for 24 hours.
(3) To 2 μl of siRNA prepared at 20 μM, 100 μl of Opti-MEM were added (1-well volume).
(4) To 4 μl of Lipofectamine 2000, 98 μl of Opti-MEM were added (1-well volume) and the whole was left to stand still at room temperature for 5 minutes.
(5) (3) and (4) were mixed to prepare a siRNA-Lipofectamin solution and left to stand still at room temperature for 20 minutes.
(6) While the solution of siRNA-Lipofectamin was left to stand still, the media in the 12-well plate were removed and 10% FBS+DMEM was added thereto in a volume of 1 ml/well.
(7) To each well, the solution of siRNA-Lipofectamin was added in a volume of 204 μl/well and the whole was incubated in 5% CO₂ at 37° C. for 24 hours.
(Each well had a final concentration of siRNA of 40 pM/1.204 ml/well.)
(8) The cells were detached with trypsin to prepare a cell solution.

### 1-2) Extraction of Total RNA

(1) The cell solution was homogenized with QIA shuredder™ (50) at 15,000 rpm×2 min.
(2) After homogenization, 350 μl of a 70% ethanol aqueous solution was added, and after pipetting, the resultant was added to RNeasy Mini Kit (50) column, followed by centrifugation at 10,000 rpm×15 sec.
(3) Further, after the column was washed with 350 μl of BufferRW1, DNase treatment was performed by using RNase-Free DNase set (50).
(4) The treatment after the column was washed with 350 μl of BufferRW1 was performed in accordance with the protocol of RNeasy Mini Kit (50).
(5) Total RNA was collected with 50 μl of RNase Free H₂O and then quantified by Gene Quant Pro (Amersham Biosciences).
1-3) Reverse transcription reaction
(1) Total RNA prepared at 100 ng/μl was used to prepare a solution for a reverse transcription reaction as follows.

| | |
|---|---|
| 2 × RT Reaction Mix | 10 μl |
| RT Enzyme Mix | 2 μl |
| RNA (100 ng/μl) | 1 μl |
| DEPC-treated Water | up to 20 μl |

(2) The reaction solution was used to carry out the reverse transcription reaction with Thermal Cycler (PERKIN ELMER) under the following condition, thereby prepare cDNA.

| | |
|---|---|
| 25° C. | 10 min |
| 42° C. | 50 min |
| 85° C. | 5 min |
| Chilled on ice | |
| 37° C. | for 20 min |

### 1-4) Real-time PCR

cDNA prepared in the previous step was used as a template to prepare a PCR reaction solution. As a positive control, Actin-β primer was used. The real-time PCR was performed with 7500 Real Time PCR system (Applied Biosystems) at n=2. The PCR condition is as follows.

### Stage 1: 95.0° C., 15 min

Reps 1

Stage 2: 94.0° C./0.15 min, 55° C./0.30 min, 72.0° C./0.34 min

Reps 45

### Sample Volume (μl): 20

Run mode: 9600 Emulation

### Data Collection: Stage2, Stage3

<Results>

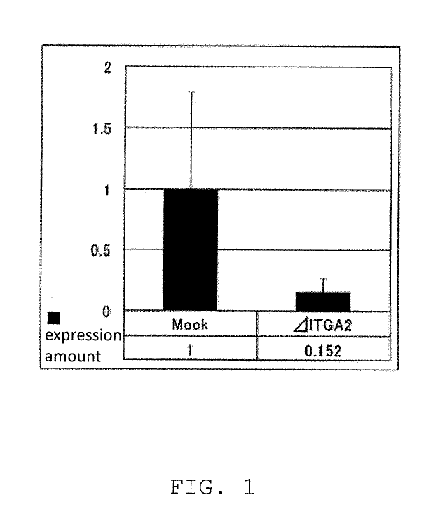

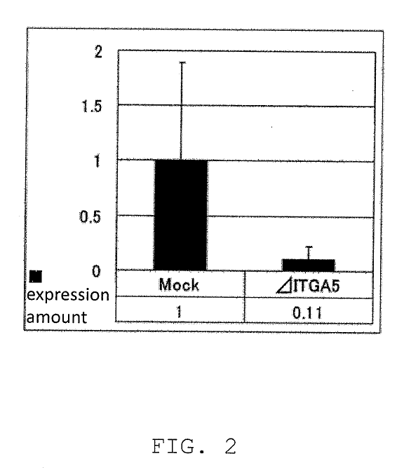

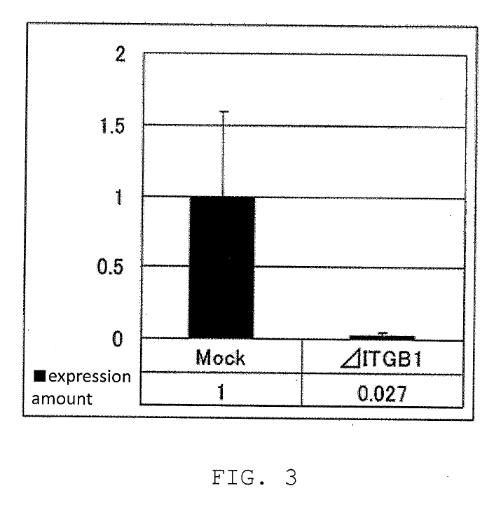

In all the knockdown strains, the expression amounts of integrin were suppressed. There is no significant difference in knockdown efficiency between siRNAs targeting the same integrin gene. However, in the knockdown strain with (2) in ITGA2, (5) in ITGA5, and (1) in ITGB1, the expression amount of integrin was most remarkably suppressed. The ratio of the expression amount of ITGA2(2) (hereinafter, ITGA2), ITGA5(5) (hereinafter, ITGA5), and ITGB1(1) (hereinafter, ITGB1) to the expression amount of the positive control determined by the real-time PCR are shown in FIGS. 1 to 3. As can be seen from the graphs, it was confirmed that the integrin gene was knocked down with siRNA in each knockdown strain. Those knockdown strains were used in the examinations below.

<Effect of Knockdown Strain on Cell Shape>

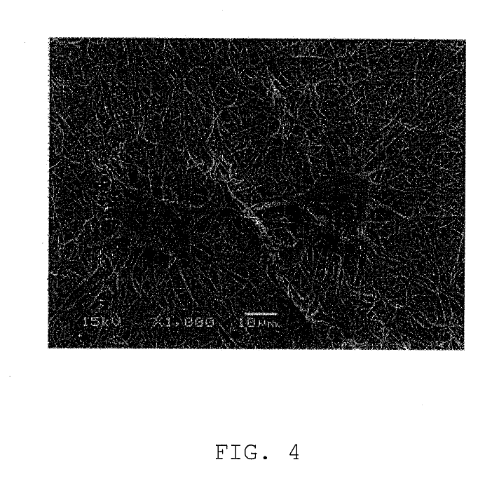

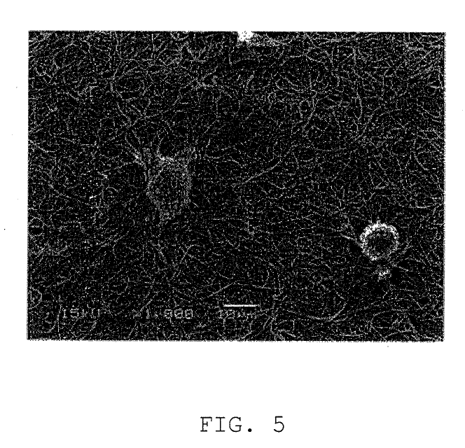

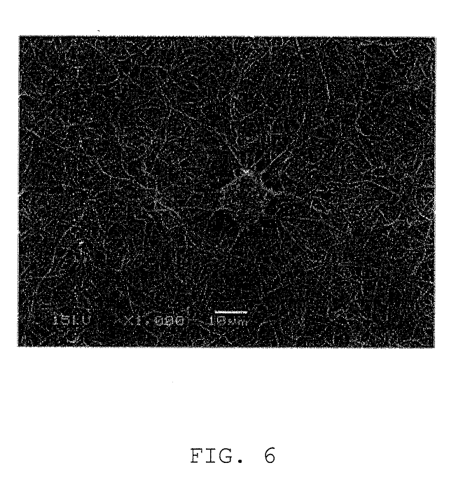

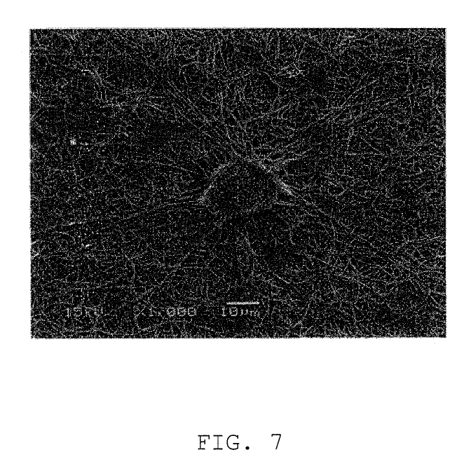

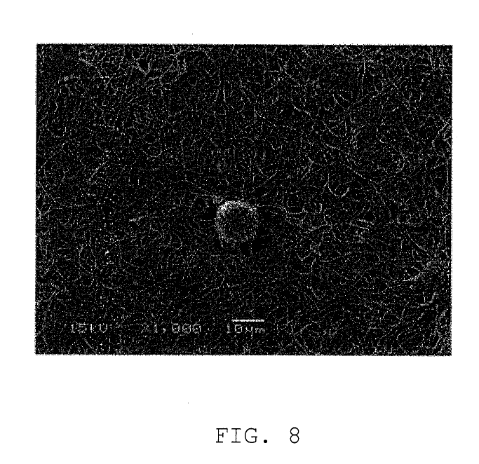

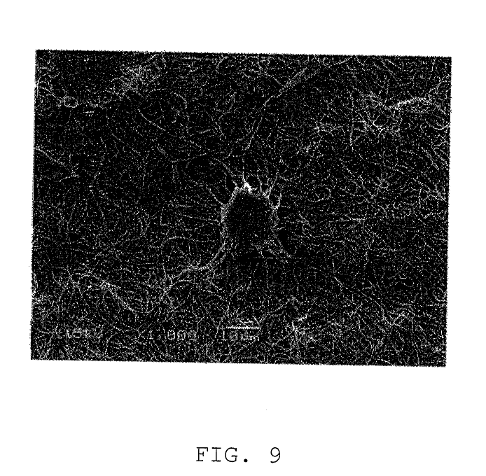

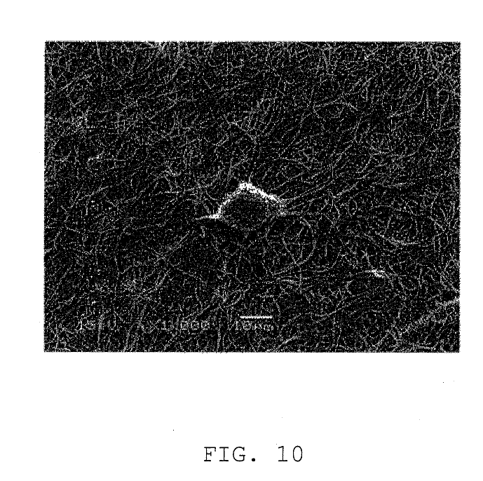

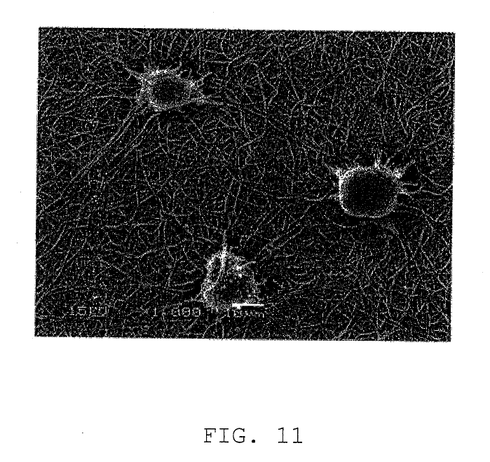

Each knockdown strain of MOCK, ITGA2, ITGA5, and ITGB1 was seeded onto a collagen gel. The cell shapes were observed by SEM after 1 and 3 hours of cell seeding. The results are shown in FIGS. 4 to 11. From those results, MOCK drew collagen fibers after 1 hour of cell seeding, and the cell shape was also flatly adhered onto the surface of the gel (FIG. 4). It was confirmed that, after 3 hours, the cells became flattened more significantly, elongated their cell projection in a stellate fashion, and drew a wider range of collagen fibers via the cell projection (FIG. 5).

On the other hand, each knockdown cell strain of ITGA2, ITGA5, and ITGB1 had more globular cell shape compared with MOCK, and also had a smaller degree of drawing collagen fibers compared with MOCK.

The foregoing demonstrated that the lower expression amount of integrin led to inhibition of tightness in collagen fiber bundles.

There is known that decrease in collagen fiber bundles affects wrinkle formation and loss in the skin elasticity. Therefore, the skin condition such as loss in the skin elasticity, wrinkle formation possibility, or insufficient tightness in collagen fiber bundles may be assessed by using the expression amount of integrin as an index.

### Example 2

<Effect of Farrerol on Collagen Fiber Bundles>

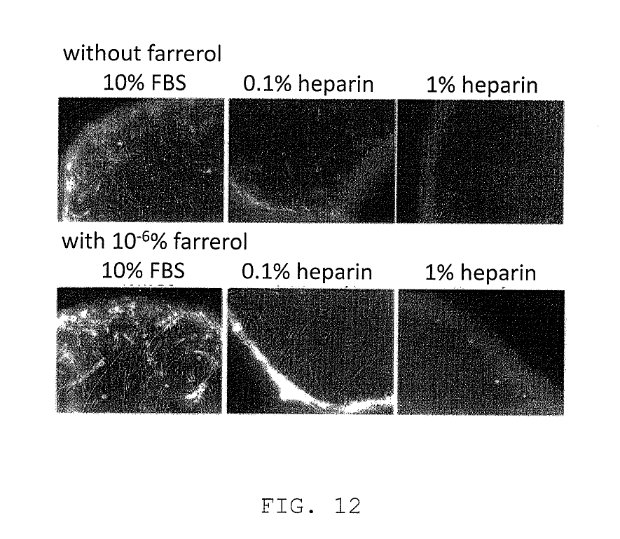

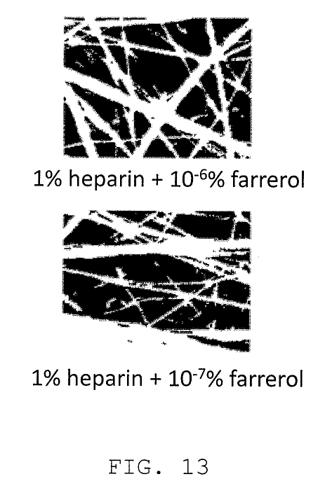

Heparin has an inhibitory effect on tightness in collagen fiber bundles. The inventors utilized the effect to screen a compound having an inhibitory effect on tightness in collagen fiber bundles. As a result, the inventors have found that farrerol had such an effect. It was studied in accordance with the following procedure that farrerol had an effect of suppressing inhibition of tightness in collagen fiber bundles due to addition of heparin. The results are shown in FIGS. 12 and 13. From those results, it was confirmed that farrerol suppressed inhibition of tightness of collagen fiber bundles due to heparin.

<Protocol>

A collagen solution was prepared by mixing 0.5% type-I collagen: 5×DMEM:200 mM HEPES: 2.2% NaHCO₃: 0.1 N NaOH: FBS: water at a ratio of 4:4:2:2:1:2:3 (while being cooled).

A lower-layer collagen solution was prepared by mixing the collagen solution: water at a ratio of 9:1.

The lower-layer collagen solution was dispensed into the respective wells of a 48-well plate in a volume of 100 μl/well.

The lower-layer collagen solution was solidified into gel in a CO₂ incubator (for about 15 minutes).

NHDF (cells) were cultivated in the same manner as in Example 1 and collected in accordance with a conventional method.

(The cells were separated from one another through a cell strainer.)

The number of the cells was counted (using Trypan blue) and then adjusted to 1×10⁵ cells/ml.

The collagen solution was mixed with the cell suspension at a ratio of 9:1 and then dispensed into the respective wells in a volume of 300 μl/well.

(For preventing the cells from uneven dispersion, the operation proceeded while intermittently stirring (1×10⁴ cells/ml at final).

The cells in the 48-well plate were left to stand still in a CO₂ incubator for 4 hours.

The collagen gel was separated from the inner wall of the well with an injection needle not to prevent the gel from contraction.

1.8% Heparin/10% FBS-DMEM was added in a volume of 500 μl/well (0.1% Heparin/well or 1% Heparin/well at final). As a control, 10% FBS-DMEM was added in a volume of 500 μl/well.

Farrerol (dissolved in DMSO) was added in a volume of 0.9 μl/well (1,000-fold dilution, 10⁻⁶% or 10⁻⁷% at final).

Cultivation was carried out in a CO₂ incubator (for 5 days).

A sample for SEM observation was obtained.

<Effect of Farrerol on Expression Amount of Integrin>

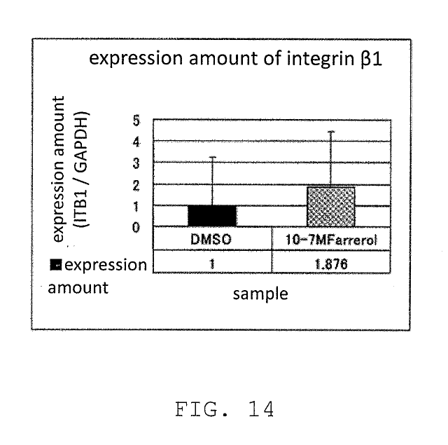

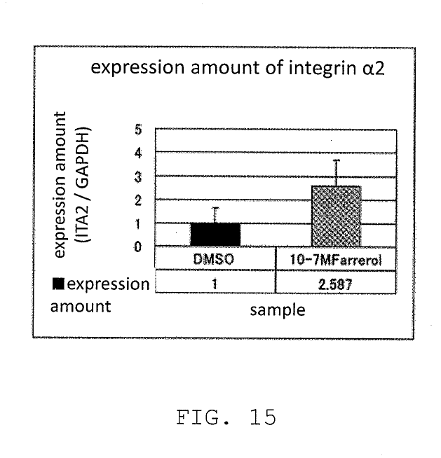

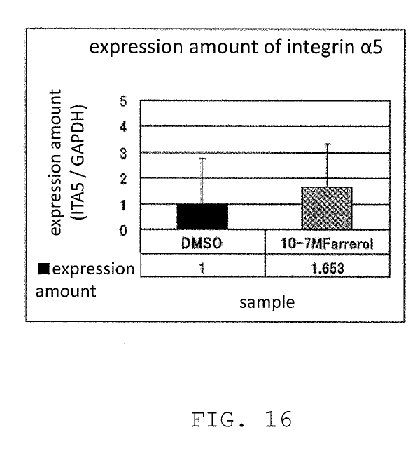

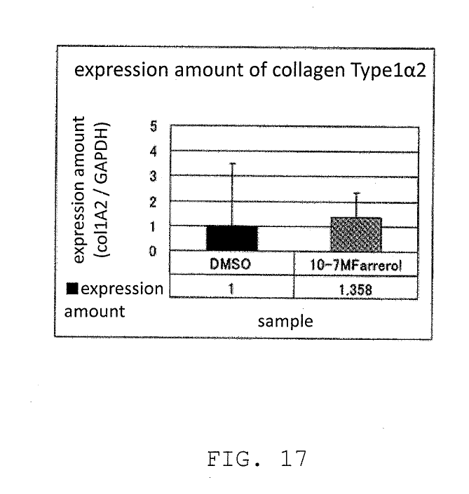

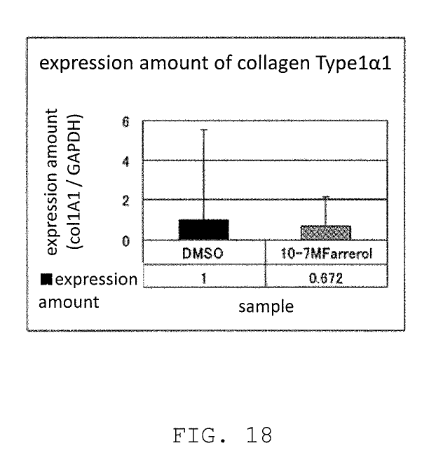

In the normal human fibroblasts cultivated in a culture solution with or without farrerol for 5 days, the expression amounts of the integrin gene and the type-I collagen gene as a main component of dermal collagen were evaluated by the real-time PCR. The procedure followed the procedure in Example 1 except for the used primer. It should be noted that the following QuantiTect (registered trademark) Primer Assays was used as a primer. The results are shown in FIGS. 14 to 18. The addition of farrerol resulted in increase in the expression amount of the integrin gene by a factor of 1.5 or more. Specifically, the expression amount of the β1 protein gene increased by a factor of about 1.9, the expression amount of the α2 protein gene increased by a factor of about 2.6, and the expression amount of the α5 protein gene increased by a factor of about 1.7. On the other hand, the expression amount of the type-I collagen gene is not significantly increased.

GAPDH (QIAGEN: Cat. No. QT00079247)

COL1A1 (QIAGEN: Cat. No. QT00037793)

COL1A2 (QIAGEN: Cat. No. QT00072058)

ITGA2 (QIAGEN: Cat. No. QT00086695)

ITGA5 (QIAGEN: Cat. No. QT00080871)

ITGB1 (QIAGEN: Cat. No. QT00068124)

<Overview>

The above-mentioned examination revealed that farrerol had an effect of increasing the expression amount of integrin in the fibroblasts. As a result of considering the above effect and the effect of farrerol which has already been confirmed by the inventors, it is revealed that increase in the expression amount of integrin linked to an effect of promoting tightness in collagen fiber bundles. There is known that decrease in collagen fiber bundles links to wrinkle formation and loss in skin elasticity. Accordingly, it is revealed that a skin condition such as loss in skin elasticity, wrinkle formation possibility, or insufficient tightness in collagen fiber bundles can be assessed by measuring the expression amount of integrin. It is also revealed that, by using difference in the expression amount of integrin in the presence and absence of a test substance, the test substance can be evaluated for its wrinkle-improvement effect.

### Example 3

<Integrin Localization Evaluation on Integrin Knockdown Strain by Fluorescent Antibody Staining Method>

In this examination, the amount and localization of an integrin α5 protein in the ITGA5 knockdown strain were visually captured, and compared with MOCK (control). That is, the expression of the integrin α5 protein was actually confirmed in the strain including the integrin gene (ITGA5) knocked down by siRNA transfection, and the localization of the integrin α5 protein in the ITGA5 knockdown strain was evaluated by the fluorescent antibody staining method.

<Cells and Reagents Used>

1) Cell culture

Normal human fibroblasts (KURABO)

10% FBS+DMEM, 2% FBS+DMEM (GIBCO)

2) siRNA

DHARMACON: 20 μM siRNA ITGA5/D-008003-05

3) Culture plate

LAB-Tek Chamber Slide w/Cover Glass Slide Sterile (NUNC: 177399)

4) Antibody staining

Primary antibody
Rabbit Anti-Integrin alpha5 Polyclonal Antibody (CHEMICON: AB1928)

Secondary antibody
Donky Anti-Rabbit IgG Rhodamine Conjugate (CHEMICON: AP182R)

Phalloidin Alexa Flour 488 (FITC) (Molecular Probes)

DAPI (Molecular Probes: D-21490)

5) Sealing agent

CRYSTAL/MAUNT (biomeda)

<Protocol>

1) To normal human fibroblasts, siRNA was transfected by lipofection. Simultaneously, MOCK was prepared as a control. The operation was performed in accordance with that of Example 1.
2) After 48 hours of siRNA transfection, the cells were detached with 0.25% Trypsin/EDTA, seeded into an 8-well slide glass chamber in a volume of 5×10³ cells/well, and samples were harvested at the time of 3, 6, 15, and 24 hours after seeding.
3) The medium was removed from the sample, followed by twice washing with PBS. The cells were immobilized by treating with a 3% paraformaldehyde/PBS at normal temperature for 5 minutes.
4) The cells were washed with PBS. 0.2% Triton-100/PBS was added, followed by treatment at normal temperature for 15 minutes.
5) The cells were washed with PBS. The primary antibody diluted with 0.1% BSA/PBS by a factor of 1,000 was added in a volume of 300 μl/well, followed by treatment at normal temperature for 60 minutes.

Further, as a negative control, only 0.1% BSA/PBS was added, followed by treatment at normal temperature for 60 minutes.

6) The cells were washed with PBS twice. The secondary antibody diluted with PBS by a factor of 500 to 1,000 was added in a volume of 300 μl/well, followed by treatment at normal temperature for 45 minutes.
7) The cells were washed with PBS. Phalloidin was added, followed by treatment at normal temperature for 20 minutes.
8) The cells were washed with PBS. DAPI was added, followed by treatment at normal temperature for 5 minutes.
9) The cells were washed with PBS. The cells were sealed with a cover glass and a sealing agent and fluorescent observation was performed with a microscope.
At the time of 24 and 48 hours after siRNA transfection, Total RNA and protein were extracted, each of which was confirmed for the knockdown efficiency of the integrin gene (see Examples 1 and 2) and the decreasing rate of the expression amount of the protein by real-time PCR and Western Blotting siRNA. The expression amount of the protein was determined by using Scion Image.

<Results>

The expression amount of integrin in the ITGA5 knockdown strain used in this examination was measured by the real-time PCR in accordance with the procedure described in Example 1. As a result, at the time of 24 hours, the expression amount was suppressed to 40% or lower compared with MOCK. Further, the expression amount of a protein in the knockdown strain was measured. As a result, at the time of 48 hours, the expression amount was suppressed to about 40% compared with MOCK. This indicates that the knockdown efficiency (decreasing rate of the transcription amount) of the integrin gene is proportional to the decreasing rate of the expression amount of the integrin protein.

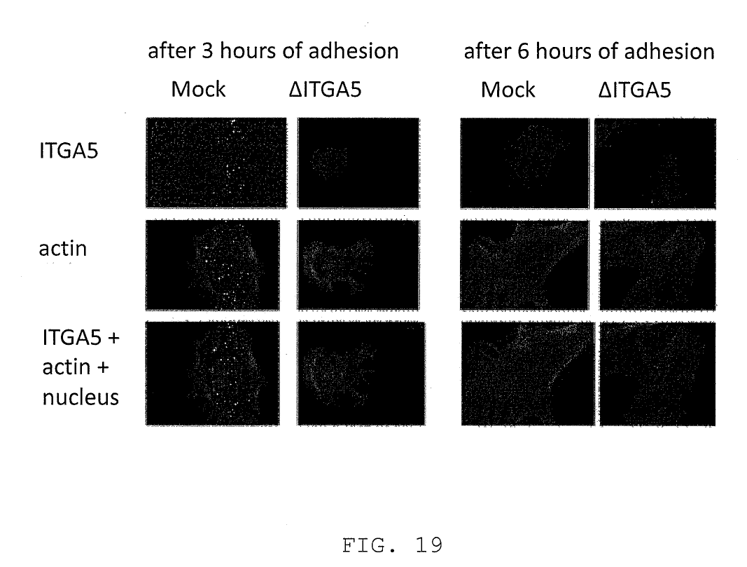

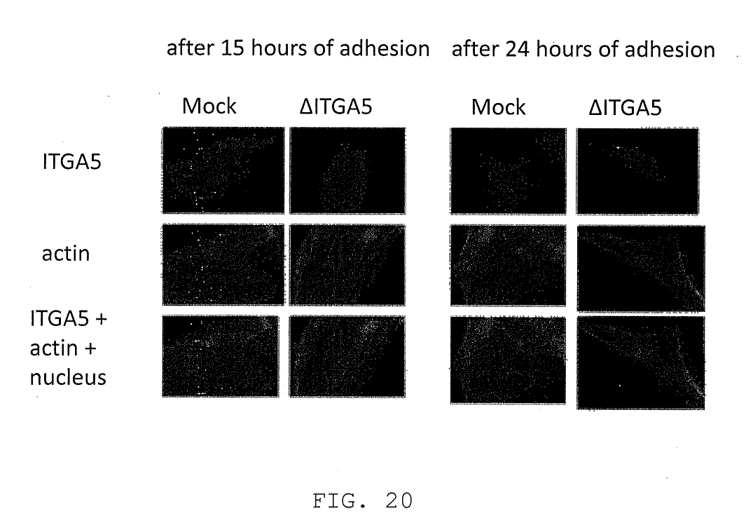

FIGS. 19 and 20 show the behavior of integrin in the cells, respectively. At the time of 3 and 6 hours after adhesion, MOCK is not significantly different from the ITGA5 knockdown strain in the behavior of the integrin α5 protein. However, after 15 hours or later, a granular integrin a5 protein is observed in MOCK, while the expression of the same granular integrin α5 protein as observed in MOCK was not observed in the ITGA5 knockdown strain.

### INDUSTRIAL APPLICABILITY

According to the method of evaluating an antiwrinkle substance of the present invention, a variety of substances can be evaluated for a wrinkle-improvement effect exactly and conveniently, which promotes the development of external preparations for skin useful for wrinkle improvement. Further, according to the method of assessing the skin of the present invention, the skin condition can be exactly and conveniently assessed, and in particular, the loss in skin elasticity, wrinkle formation possibility, insufficient tightness in dermal collagen fiber bundles, or the like can be exactly and conveniently evaluated.

1. A method of evaluating an antiwrinkle substance, comprising evaluating a test substance for its wrinkle-improvement effect, comprising measuring an expression amount of an adhesion factor in an animal cell in the presence of the test substance.

2. A method of evaluating an antiwrinkle substance according to claim 1, comprising evaluating a test substance for its wrinkle-improvement effect, comprising measuring the degree of difference in expression amounts of the adhesion factor in an animal cell in the presence and absence of the test substance.

3. A method of evaluating an antiwrinkle substance according to claim 2, comprising evaluating a test substance exhibiting a larger difference in expression amounts of the adhesion factor to have a larger wrinkle-improvement effect.

4. A method of evaluating an antiwrinkle substance according to claim 1, comprising evaluating a test substance in which the expression amount of the adhesion factor in the presence of the test substance is 1.5 or more times higher than the expression amount of the adhesion factor in the absence of the test substance to have a larger wrinkle-improvement effect.

5. A method of evaluating an antiwrinkle substance according to claim 1, wherein the adhesion factor is integrin.

6. A method of assessing a skin condition, comprising measuring an expression amount of an adhesion factor in a skin cell.

7. A method of assessing a skin condition according to claim 6, comprising assessing skin exhibiting a lower expression amount of the adhesion factor to be in poorer condition.

8. A method of assessing a skin condition according to claim 7, comprising assessing skin exhibiting the lower expression amount of the adhesion factor to have a larger loss in elasticity.

9. A method of assessing a skin condition according to claim 7, comprising assessing skin exhibiting the lower expression amount of the adhesion factor to have a higher wrinkle formation possibility.

10. A method of assessing a skin condition according to claim 7, comprising assessing skin exhibiting the lower expression amount of the adhesion factor to have a more insufficient tightness in dermal collagen fiber bundles.

11. A method of assessing a skin condition according to claim 6, wherein the adhesion factor is integrin.

12. An external preparation for skin, comprising an antiwrinkle substance evaluated to have a larger wrinkle-improvement effect by the method of evaluating an antiwrinkle substance according to claim 4.

13. A method of producing an external preparation for skin, comprising: evaluating a wrinkle-improvement effect by the method of evaluating an antiwrinkle substance according to claim 4; and formulating an antiwrinkle substance evaluated to have a larger wrinkle-improvement effect into an external preparation for skin.
